(19) United States
(12) Patent Application Publication
(10) Pub. No.: US 2022/0162240 A1
(43) Pub. Date: May 26, 2022

(54) MITOCHONDRIA-TARGETED ISOKETAL/ISOLEVUGLANDIN SCAVENGERS

(71) Applicant: Vanderbilt University, Nashville, TN (US)

(72) Inventors: Sergey I Dikalov, Nashville, TN (US); Venkataraman Amarnath, Nashville, TN (US)

(21) Appl. No.: 17/425,894

(22) PCT Filed: Jan. 27, 2020
(86) PCT No.: PCT/US2020/015197

§ 371 Date: Jul. 26, 2021

Related U.S. Application Data
(60) Provisional application No. 62/796,889, filed on Jan. 25, 2019.

(51) Int. Cl. C07F 9/54; C07D 213/04; C07C 229/34

(52) U.S. Cl. CPC C07F 9/5456; C07C 229/34; C07D 213/04

(57) ABSTRACT

Compounds and methods for treating, preventing, and ameliorating at least one of vascular oxidative stress, improve vascular functions and/or reduce hypertension, comprising administering to a subject a compound that targets mitochondrial CypD to inhibit vascular oxidative stress, improve vascular functions and/or reduce hypertension.

### GOVERNMENT SUPPORT

None.

### BACKGROUND OF THE INVENTION

The present invention relates to mitochondria-targeted scavengers of highly reactive lipid dicarbonyls derived from arachidonic acid and other polyunsaturated fatty acids, isolevuglandins (isoLG, also known as isoketals or gamma-ketoaldehydes), pharmaceutical compositions comprising such compounds, and methods of treating conditions involving inflammation, oxidative stress, and/or mitochondrial dysfunction.

One aspect of the present invention is novel mitochondria-targeted compounds. Without being bound by mechanism or theory, these compounds are not typical antioxidants, but they scavenge the products of inflammation and protect endothelium-dependent relaxation.

Highly reactive lipid dicarbonyls such as isoLG causes cell dysfunction, cytotoxic and immunogenic, promoting inflammation and tissue damage in cardiovascular diseases, hypertension, cancer, and neurodegeneration.

Cardiovascular diseases and cancer are the main causes of death in Western Societies. In 2002, over 450,000 Americans under 85 died of cancer and died of heart disease. The present invention meets a long-felt need for treatment of both cardiovascular diseases and cancer based on contribution of oxidative stress in both pathological conditions.

Approximately 50 million people in the United States have overt hypertension, and up to 60% of the population is pre-hypertensive. This is a major health care concern because hypertension markedly increases the risk of death from stroke, ischemic heart disease, and other vascular diseases. An important mediator is the hormone angiotensin II, which increases thirst, promotes salt retention by the kidney, causes vasoconstriction, and enhances the release of catecholamines from nerves and the adrenal gland. Angiotensin II also directly promotes inflammation and the development of atherosclerosis. Hypertension is linked to the vascular oxidative stress and accumulation of reactive lipid dicarbonyls such as isolG. Scavenging of reactive lipid dicarbonyls with salicylamine (2-HOBA) attenuates hypertension in animal studies, prevents inflammation and protects endothelium-dependent relaxation.

### SUMMARY OF THE INVENTION

One embodiment of the present invention is compounds that are mitochondria-targeted scavengers of lipid dicarbonyls.

In one embodiment, the compound is of the following formula:

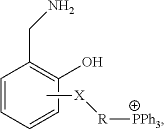

wherein:
X is a bond, —O—, or —CH₂—; and
R is C₁ to C₁₂ substituted or unsubstituted alkyl; and stereoisomers and pharmaceutical salts thereof.

In another embodiment, the mitochondria-targeted scavenger is a compound of the following formula:

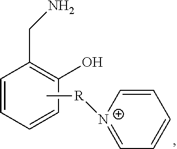

wherein
R is C₁ to C₁₂ substituted or unsubstituted alkyl; and stereoisomers and pharmaceutical salts thereof.

In another embodiment, the mitochondria-targeted scavenger is a compound of the following formula:

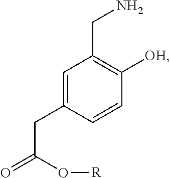

wherein
R is C₁ to C₁₂ substituted or unsubstituted alkyl; and stereoisomers and pharmaceutical salts thereof.

In another embodiment, the mitochondria-targeted scavenger is a compound of the following formula:

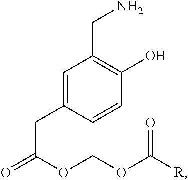

wherein
R is C₁ to C₁₂ substituted or unsubstituted alkyl; and stereoisomers and pharmaceutical salts thereof.

In another embodiment, the mitochondria-targeted scavenger is a compound of the following formula:

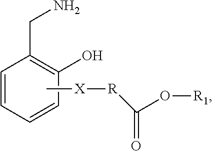

wherein

X is a bond, —O—, or —CH₂—;

R is C₁ to C₁₂ substituted or unsubstituted alkyl; and

R₁ is C₁ to C₁₂ substituted or unsubstituted alkyl or acetoxymethyl; and stereoisomers and pharmaceutical salts thereof.

In another embodiment, the mitochondria-targeted scavenger is a compound of the following formula:

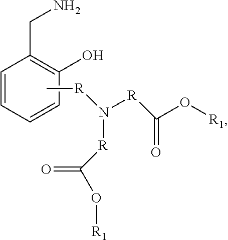

wherein

each R is independent and chosen from C₁ to C₁₂ substituted or unsubstituted alkyl; and

each R₁ is independent and chosen from C₁ to C₁₂ substituted or unsubstituted alkyl or acetoxymethyl; and stereoisomers and pharmaceutical salts thereof.

In another embodiment, the mitochondria-targeted scavenger is a compound of the following formula:

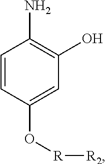

wherein
R is C₁ to C₁₂ substituted or unsubstituted alkyl; R₂ is selected from —P-Ph₃; or

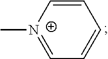

and stereoisomers and pharmaceutical salts thereof.

In yet another embodiment, the compound is of the following formula:

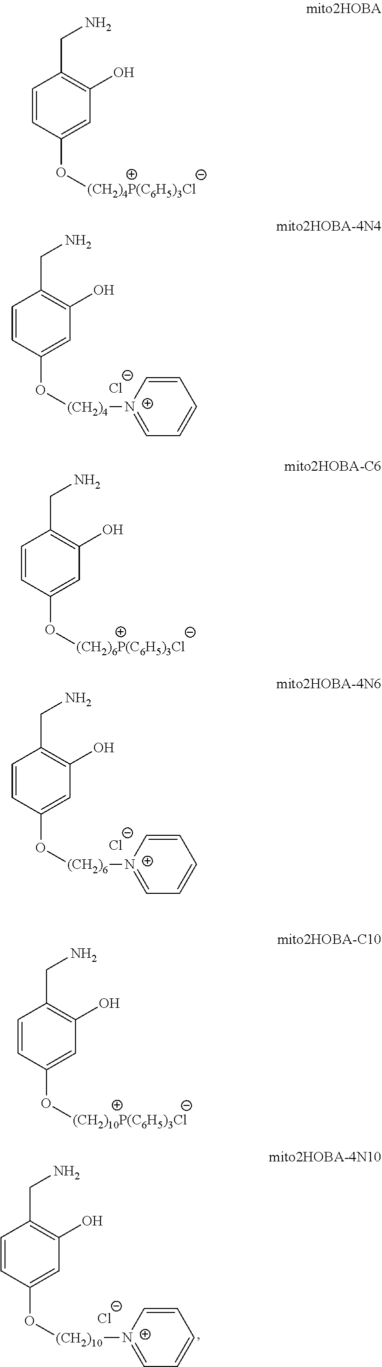

and stereoisomers and pharmaceutical salts thereof.

In another embodiment of the present invention, a method is provided for treating, preventing, and ameliorating hypertension in a subject, comprising administering an effective amount of a mitochondria-targeted scavenger of the present invention, or a pharmaceutically acceptable salt thereof.

In another embodiment of the present invention, a method is provided for treating, preventing, and ameliorating vascular oxidative stress in a subject, comprising administering an effective amount of a mitochondria-targeted scavenger of the present invention, or a pharmaceutically acceptable salt thereof.

Another embodiment of the present invention is a method of treating, preventing, and ameliorating at least one of vascular oxidative stress, improve vascular functions and/or reduce hypertension, comprising administering to a subject a compound that targets mitochondrial CypD to inhibit vascular oxidative stress, improve vascular functions and/or reduce hypertension.

Another embodiment of the present invention a compound of the following formula:

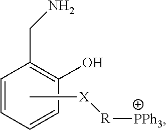

wherein:
X is a bond, —O—, or —CH₂—; and
R is C₁ to C₁₂ substituted or unsubstituted alkyl; and stereoisomers and pharmaceutical salts thereof for use in treating, preventing, and ameliorating hypertension in a subject,

Another embodiment of the present invention is a compound of the following formula:

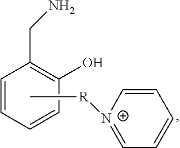

wherein
R is C₁ to C₁₂ substituted or unsubstituted alkyl; and stereoisomers and pharmaceutical salts thereof, for use in treating, preventing, and ameliorating hypertension in a subject.

Another embodiment of the present invention is a compound of the following formula:

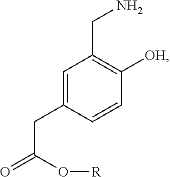

wherein
R is C₁ to C₁₂ substituted or unsubstituted alkyl; and stereoisomers and pharmaceutical salts thereof; for use in treating, preventing, and ameliorating hypertension in a subject,

Another embodiment of the present invention is a compound of the following formula:

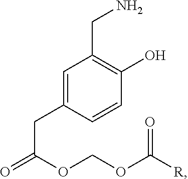

wherein
R is C₁ to C₁₂ substituted or unsubstituted alkyl; and stereoisomers and pharmaceutical salts thereof, for use in treating, preventing, and ameliorating hypertension in a subject,

Another embodiment of the present invention is a compound of the following formula:

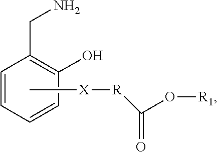

wherein
X is a bond, —O—, or —CH₂—;
R is C₁ to C₁₂ substituted or unsubstituted alkyl; and
R₁ is C₁ to C₁₂ substituted or unsubstituted alkyl or acetoxymethyl; and stereoisomers and pharmaceutical salts thereof; for use in treating, preventing, and ameliorating hypertension in a subject.

Another embodiment of the present invention is a compound of the following formula:

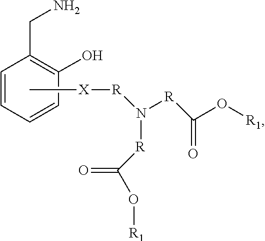

wherein
each R is independent and chosen from C₁ to C₁₂ substituted or unsubstituted alkyl; and
each R₁ is independent and chosen from C₁ to C₁₂ substituted or unsubstituted alkyl or acetoxymethyl; and stereoisomers and pharmaceutical salts thereof; for use in treating, preventing, and ameliorating hypertension in a subject.

In another embodiment, the mitochondria-targeted scavenger is a compound of the following formula:

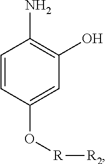

wherein
R is C₁ to C₁₂ substituted or unsubstituted alkyl; R₂ is selected from —P-Ph₃; or

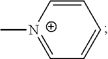

and stereoisomers and pharmaceutical salts thereof; for use in treating, preventing, and ameliorating hypertension in a subject.

Another embodiment of the present invention is a compound is of the following formula:

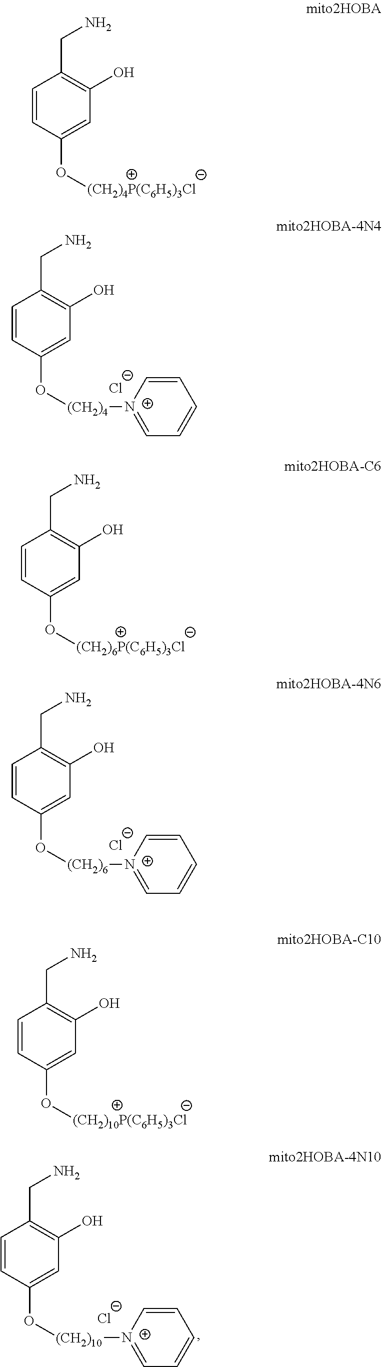

and stereoisomers and pharmaceutical salts thereof; for use in treating, preventing, and ameliorating hypertension in a subject.

### BRIEF DESCRIPTION OF THE DRAWINGS

FIG. 1 is schematic diagram showing CypD hyperacetylation, vascular oxidative stress, and hypertension. The present inventors have discovered that CypD hyperacetylation promotes vascular oxidative stress and contributes to hypertension, and that measures to reduce CypD acetylation and CypD inhibition will improve vascular function and attenuate hypertension.

FIG. 2A-2D is a set of graphs showing angiotensin II-induced hypertension (A), vascular mitochondrial [symbol] (B), and vasodilatation of aortic vessels with acetylcholine (C) or NO-donor SNP (D). Blood pressure was measured by telemetry. Following 14 days of saline or Ang II (0.7 mg/kg/day) infusion, mice were sacrificed for isolation of aorta to study mitochondrial [symbol] and vasodilation. *P<0.01 vs Sham, **P<0.01 vs WT+Ang II, ***P<0.01 vs WT (n=8).

FIG. 3A-3D is a set of graphs showing examples of targeting CypD in hypertension. Blood pressure in C57Bl/6J mice infused with saline (Sham), Ang II (0.7 mg/kg/day) or treated with CypD blocker Sanglifehrin A (SFA) after onset of Ang II-induced hypertension (Ang II+SFA). Blood pressure was measured by tail-cuff method (A). ³Following 14 days of saline or Ang II infusion, mice were sacrificed for isolation of aorta to study mitochondrial [symbol] using MitoSOX and HPLC or to study vasodilation. Results are mean±SEM (n=6-8). *P<0.01 vs Sham, **P<0.01 vs Ang II, ***P<0.01 vs Ang II+SFA (n=8).

FIG. 4A-4D is a set of graphs showing mitochondrial O₂\dot{—} (A) and vasorelaxation (B,C,D) in vessels treated ex vivo with combination of Ang II (10 nM), IL17A (10 ng/ml) and TNFα (1 ng/ml) for 24 hours (ATI). Aortas were isolated from C57Bl/6J (WT), CypD^{-/-}, Tg^{SOD2} or mCAT mice. Mitochondrial [symbol] was measured by MitoSOX and HPLC.³ Results are mean±SEM (n=8). *P<0.01 vs WT, **P<0.05 vs WT+ATI.

FIG. 5 shows Western blot of Sirt3 expression and acetylation of mitochondrial proteins (mito Ac-K) in patients with essential hypertension compared with normotensive subjects. Results are mean±SEM (n=6). *P<0.01 vs Normotensive, **P<0.001 vs Normotensive.

FIG. 6 shows mitochondrial hyperacetylation and CypD acetylation in hypertension. Western blot of mitochondria isolated from aorta dissected from C57Bl/6J and CypD^{-/-} mice infused with Ang II. CypD acetylation was determined by CypD immunoprecipitation and Western blot with anti-Acetyl-Lysine antibody. FIG. 6 also shows representative blots from three experiments.

FIG. 7 shows that depletion of CypD or GCN5L1 acetylase prevents simulation of mitochondrial [symbol] but Sirt3 depletion leads to [symbol] overproduction. HAEC were treated with Ang II (10 ng/ml) plus TNFα (1 ng/ml) for 24 hours and mitochondrial [symbol] was measured by MitoSOX and HPLC. The figure also shows a typical CypD Western blot analysis. Results are mean±SEM (n=6). *P<0.01 vs Sham, **P<0.05 vs NS, ***P<0.05 vs NS, P<0.05 vs Sham.

FIG. 8 shows mitochondrial swelling (A) and impaired respiration (B) induced by isoLG or isoLG-PE. Intact mouse kidney mitochondria with glutamate and malate were incubated (5 min) with ethanol as vehicle, isoLG (1 μM) or isoLG-PE (1 μM) prior to addition of ADP (50 μM) and measurements of oxygen consumption. *P<0.001 vs Control, **P<0.03 vs isoLG.

FIG. 9 shows inhibition of mitochondrial oxidative stress by mito2HOBA. HAEC were treated with mito2HOBA (50 nM), 2HOBA or isoLG-inactive 4HOBA and incubated with Ang II (100 nM) plus TNFα (10 nM) for 24 hours prior to measurements of mitochondrial O₂\dot{—} by MitoSOX using HPLC (A) and cardiolipin oxidation (B) by LC/MS. *P<0.001 vs control, **P<0.01 vs AngII/TNFα.

FIG. 10 shows the effect of mito2HOBA on Ang II-induced hypertension, isoLG adducts and CypD acetylation in aortic mitochondria. (A) Blood pressure in C57Bl/6J mice infused with saline (Sham) or Ang II (0.7 mg/kg/ml). Mito2HOBA was supplemented in drinking water (0.1 g/L). (B) Western blot of aorta mitochondria for isoLG adducts (D11 antibody), CypD, GCN5L1, Sirt3 and CypD acetylation (CypD i.p. and anti-Acetyl-K WB). Results are mean±SEM. *P<0.01 vs Sham, **P<0.01 vs Ang II (n=8).

FIG. 11A-11B shows a LS/MS/MS analysis of mitochondrial isoLG-Lys-Lactam protein adducts. (A) Representative LC/MS/MS chromatograms; (B) isoLG-Lys-Lactam levels in mitochondria isolated from kidneys of mice drinking water (Sham), mito2HOBA (0.1 g/L), and infused with Ang II (0.7 mg/kg/day). Results are mean±SEM (n=3). *P<0.05 vs Ang II.

FIG. 12 shows that mito2HOBA reduces mPTP opening and prevents mitochondrial dysfunction. C57Bl/6J mice were infused with Ang II (0.7 mg/kg/ml) and mito2HOBA in the drinking water (0.1 g/L). Following 14 days of Ang II infusion the animals were sacrificed and kidneys were isolated for mitochondrial studies. Addition of CaCl₂) to mitochondria above Ca²⁺ retention capacity leads to mPTP opening and mitochondria swelling. Mitochondria isolated from Ang II-infused mice had significant reduction in Ca²⁺ capacity due to increased mPTP opening and CypD inhibitor Cyclosporine A (CsA) rescues Ca²⁺ retention capacity (A). Respiratory control ratio (State 3/State 4) was measured in isolated kidney mitochondria with glutamate and malate (B). Control level is 100%. (B) Kidney ATP was measured in freshly isolated tissue by luciferase-based luminescent assay. Results are mean±SEM. *P<0.01 vs Sham, **P<0.01 vs Ang II (n=3-8).

FIG. 13 shows the effect of mito2HOBA on aortic [symbol] (A) and endothelial NO (B) in Ang II-infused mice. (A) Aortic [symbol] was measured by DHE probe and HPLC. (B) Endothelial NO was analyzed by ESR and Fe(DETC)₂. C57Bl/6J mice were infused with Ang II (0.7 mg/kg/ml) and mito2HOBA was provided in the drinking water (0.1 g/L). Results are mean±SEM. *P<0.01 vs Sham, **P<0.01 vs Ang II (n=6).

FIG. 14 is a schematic drawing demonstrating the discovery that isolevuglandins activate CypD which contributes to mitochondrial dysfunction, vascular oxidative stress and hypertension, and that scavenging of mitochondrial isoLG will reduce endothelial dysfunction, and diminish hypertension.

FIG. 15 shows reactions of isoLG with protein lysine and phosphatidylethanolamine (PE) and scavenging of isoLG by 2-hydroxybenzylamine (2HOBA) or mitochondria-targeted analog mito2HOBA.

FIG. 16 shows the effect of mito2HOBA on angiotensin II-induced hypertension and accumulation of isoLG mitochondrial protein adducts. (A) Blood pressure in C57Bl/6J wild type mice infused with either saline (Sham) or Ang II (0.7 mg/kg/ml). Mito2HOBA was supplemented in drinking water (0.1 g/L). (B) Mitochondrial isoLG were measured by Western blot in the heart mitochondria using D11 antibody as we have previously described. Results are mean±SEM. *P<0.01 vs Sham, **P<0.01 vs Ang II (n=8).

FIG. 17 shows mitochondrial oxidative stress in hypertension. (A) Systolic blood pressure in C57Bl/6J wild-type (WT) and mCAT mice infused with saline (Sham) or Ang II (0.3 mg/kg/day); (B) measurements of cardiolipin oxidation by LC-MS.³⁶ Following 14 days of saline or Ang II infusion, mice were sacrificed for isolation of heart for measurements of cardiolipin oxidation. *P<0.01 vs Sham, **P<0.01 vs Ang II (n=6).

FIG. 18 is a western blot analysis of isoLG protein adducts in mitochondria isolated from aorta dissected from C57Bl/6J mice infused with Ang II and treated with mito2HOBA (A). CypD-isoLG modification was determined by CypD immunoprecipitation and Western blot with anti-isoLG D11 antibody (B). The figure shows representative blots from three experiments.

FIG. 19 shows mito2HOBA attenuates mitochondrial dysfunction. (A) Respiratory control ratio (State 3/State 4) was measured in isolated kidney mitochondria with glutamate and malate. Control level is 100%. (B) Kidney ATP level was measured in freshly isolated tissue by luciferase-based luminescent assay. C57Bl/6J mice were infused with Ang II (0.7 mg/kg/ml) and mito2HOBA was provided in the drinking water (0.1 g/L). Following 14 days of Ang II infusion the animals were sacrificed and kidneys were isolated for mitochondrial and ATP studies. Results are mean±SEM. *P<0.01 vs Sham, **P<0.01 vs Ang II (n=3-8).

### DESCRIPTION OF THE INVENTION

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which need to be independently confirmed.

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “optional” or “optionally” means that the subsequently described event or circumstance can or cannot occur, and that the description includes instances where said event or circumstance occurs and instances where it does not.

As used herein, the term “subject” refers to a target of administration. The subject of the herein disclosed methods can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed. As can be seen herein, there is overlap in the definition of treating and preventing.

As used herein, the term “diagnosed” means having been subjected to a physical examination by a person of skill, for example, a physician, and found to have a condition that can be diagnosed or treated by the compounds, compositions, or methods disclosed herein. As used herein, the phrase “identified to be in need of treatment for a disorder,” or the like, refers to selection of a subject based upon need for treatment of the disorder. For example, a subject can be identified as having a need for treatment of a disorder (e.g., a disorder related to inflammation) based upon an earlier diagnosis by a person of skill and thereafter subjected to treatment for the disorder. It is contemplated that the identification can, in one aspect, be performed by a person different from the person making the diagnosis. It is also contemplated, in a further aspect, that the administration can be performed by one who subsequently performed the administration.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “scavenger” or “scavenging” refers to a chemical substance that can be administered in order to remove or inactivate impurities or unwanted reaction products. For example, the isoketals irreversibly adduct specifically to lysine residues on proteins. The isoketal scavengers of the present invention react with isoketals before they adduct to the lysine residues. Accordingly, the compounds of the present invention “scavenge” isoketals, thereby preventing them from adducting to proteins.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, optionally substituted alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

Embodiments of the present invention include methods of treating, preventing, and ameliorating hypertension in a subject.

Other embodiments of the present invention include methods of treating, preventing, and ameliorating vascular oxidative stress in a subject.

Other embodiments of the present invention include methods of targeting mitochondrial CypD to inhibit vascular oxidative stress, improve vascular functions and/or reduce hypertension.

The present invention, for the first time, defines CypD as a target for treatment of hypertension. There have been no mechanistically novel treatments for this disease in the past 30 years. The antihypertensive agents of the present invention, targeting CypD, could add to the currently available therapeutic armamentarium to improve treatment of hypertension.

Free radical oxidation of arachidonic acid produces highly reactive isolevuglandins (isoLG) which causes mitochondrial dysfunction by opening of the mitochondrial permeability transition pore (mPTP). Inhibition of the mPTP regulatory subunit cyclophilin D reduces isoLG-induced mitochondrial dysfunction (Free Radic Biol Med 2010; 49(4):567-79). The present inventors found that depletion of cyclophilin D diminishes mitochondrial [symbol] , improves vascular relaxation and reduces hypertension (Hypertension 2016; 67(6):1218-27). The present inventors tested their hypothesis that hypertension is associated with accumulation of mitochondrial isoLG and mitochondria-targeted isoLG scavenger reduces vascular oxidative stress and attenuates hypertension. The present inventors developed novel mitochondria-targeted isoLG scavenger compounds, including 4-(aminomethyl)-3-hydroxyphenoxy) butyl)triphenylphosphonium (mito2HOBA) compounds by conjugation of lipophilic cation triphenylmethylphosphonium to 2-hydroxybenzylamine (2HOBA). Mito2HOBA is a water soluble compound which was well tolerated by cultured human aortic endothelial cells (HAECs) and by mice receiving it in drinking water. It was discovered that mito2HOBA (50 nM) inhibited mitochondrial [symbol] production (MitoSOX/HPLC) and prevented cardiolipin oxidation (LS-MS) in HEACs incubated with TNFα+Angiotensin II while 2HOBA (50 nM) was not effective. The functional role of mitochondrial isoLG was tested in vivo using angiotensin II model of hypertension. C57Bl/6J mice were infused with angiotensin II (0.7 mg/kg/day) or saline (Sham) and received mito2HOBA in the drinking water (0.1 g/L). It was also discovered that mito2HOBA significantly attenuated angiotensin II-induced hypertension. Hypertension was associated with increased formation of mitochondrial isoLG measured by Western blot analysis of heart mitochondria using D11 antibody and mito2HOBA reduced accumulation of isoLG adducts in the heart mitochondria of angiotensin II-infused mice. Decrease of NO is a hallmark of endothelial dysfunction in hypertension due to vascular oxidative stress.

The present inventors examined vascular oxidative stress by measurements of aortic [symbol] using fluorescent [symbol] probe DHE and HPLC, and analysis of endothelial NO using Electron Spin Resonance and specific NO spin trap Fe(DETC)₂. It was discovered that mito2HOBA reduced vascular [symbol] in angiotensin II-infused mice and preserved endothelial NO.

2-Aminomethylphenols, exemplified by 2-hydroxybenzylamine (2-HOBA, salicylamine) exhibit extraordinary reactivity towards disease causing dicarbonyls.

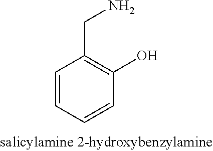

Mitochondria, being the site of oxidation, can be damaged by products of oxidative stress and it is attributed to many diseases, including multiple sclerosis. While 2-HOBA can be expected to scavenge these reactive molecules and afford protection, it needs to be modified for access into mitochondria. Certain cationic attachments have been successfully used in the past to accomplish this.

Embodiments of the present invention include series of mitochondria-targeted compounds. One embodiment of the present invention is a compound of the following formula:

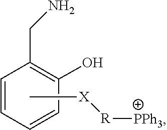

wherein:
X is a bond, —O—, or —CH₂—; and
R is C₁ to C₁₂ substituted or unsubstituted alkyl; and stereoisomers and pharmaceutical salts thereof.

Another embodiment of the present invention is a compound of the following formula:

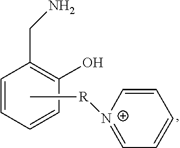

wherein
R is C₁ to C₁₂ substituted or unsubstituted alkyl; and stereoisomers and pharmaceutical salts thereof.

Another embodiment of the present invention is a compound of the following formula:

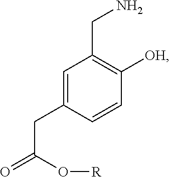

wherein
R is C₁ to C₁₂ substituted or unsubstituted alkyl; and stereoisomers and pharmaceutical salts thereof.

Another embodiment of the present invention is a compound of the following formula:

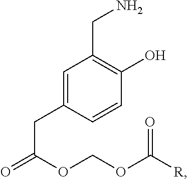

wherein
R is C₁ to C₁₂ substituted or unsubstituted alkyl; and stereoisomers and pharmaceutical salts thereof.

Another embodiment of the present invention is a compound of the following formula:

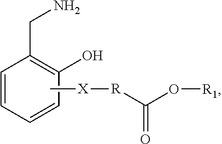

wherein
X is a bond, —O—, or —CH₂—;
R is C₁ to C₁₂ substituted or unsubstituted alkyl; and
R₁ is C₁ to C₁₂ substituted or unsubstituted alkyl or acetoxymethyl; and stereoisomers and pharmaceutical salts thereof.

Another embodiment of the present invention is a compound of the following formula:

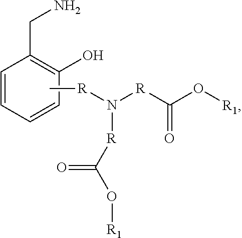

wherein
each R is independent and chosen from C₁ to C₁₂ substituted or unsubstituted alkyl; and
each R₁ is independent and chosen from C₁ to C₁₂ substituted or unsubstituted alkyl or acetoxymethyl; and stereoisomers and pharmaceutical salts thereof.

Another embodiment of the present invention is to use lipophilic esters (preferably acetoxymethyl) to transport the ‘pro-drug’ across the membrane and hydrolysis by intracellular esterase release ionic acid that is trapped inside the cell compartment.

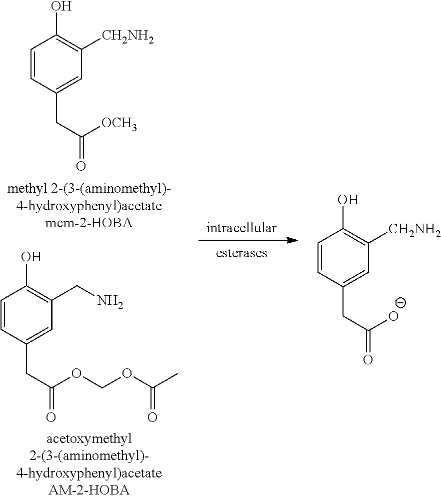

Another aspect of the invention is additional compounds that on hydrolysis increase the ionic nature of the resulting 2-HOBA.

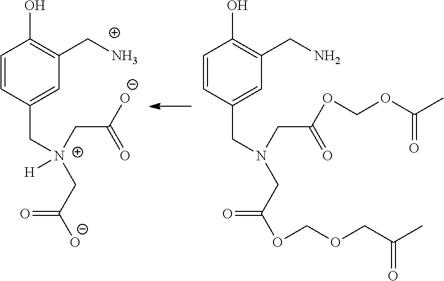 [SMILES: NCc1cc(C[NH+](CC(=O)[O-])CC(=O)[O-])ccc1O ; CC(=O)COCOC(=O)CN(CC(=O)OCOC(C)=O)Cc1ccc(O)c(CN)c1]

Another embodiment of the present invention is a compound of the following formula:

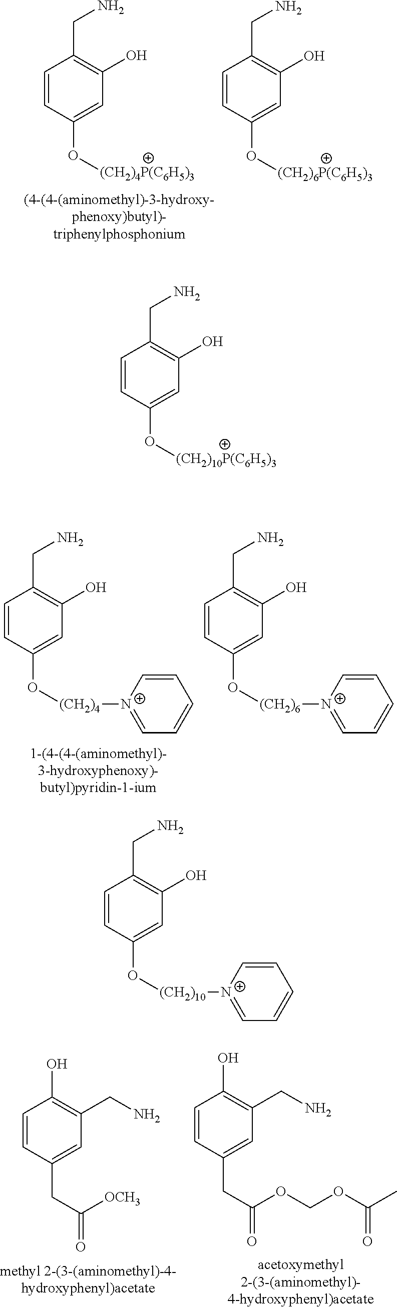

and stereoisomers and pharmaceutical salts thereof.

Examples of the present invention include the following compounds:

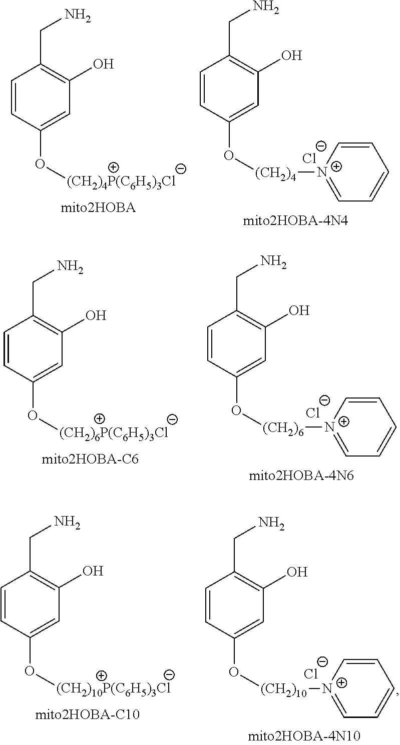

and stereoisomers and pharmaceutical salts thereof.

### I. Hypertension

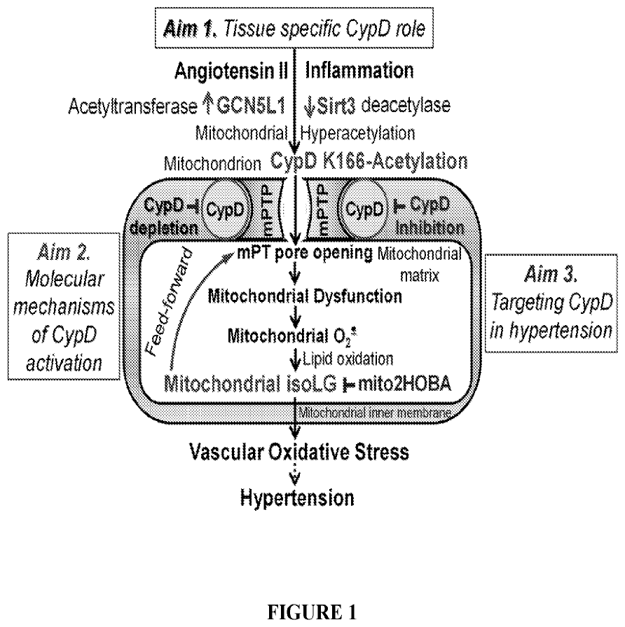

Hypertension is a major health problem in Western Societies and a risk factor for stroke, myocardial infarction, and heart failure. Blood pressure is poorly controlled in third of patients despite use of multiple drugs, likely due to mechanisms contributing to hypertension unaffected by current treatments. In the past several years, we have shown that the mitochondria are dysfunctional in hypertension and have defined a novel role of mitochondrial superoxide ( [symbol] ) in this disease. The present inventors have shown a key role of mitochondrial cyclophilin D (CypD) in stimulation of mitochondrial [symbol] by angiotensin II and cytokines. CypD is a regulatory subunit of the mitochondrial permeability transition pore (mPTP). The present inventors found that genetic CypD depletion attenuates hypertension while treatment with CypD inhibitor after onset of hypertension reduces blood pressure. An aspect of the present invention is defining the role of vascular CypD and therapeutic potential of targeting CypD in vascular dysfunction and hypertension. The present inventors' studies in animals and human subjects with essential hypertension implicate CypD activation by K166 acetylation due to imbalance between GCN5L1 acetyltransferase and reduced Sirt3 deacetylase activity. Sirt3 level is reduced in hypertension and residual Sirt3 is blocked by highly reactive mitochondrial lipid dicarbonyls, isolevuglandins (isoLG), while isolG scavenging prevents CypD hyperacetylation and reduces hypertension (FIG. 1).

The present invention defines CypD as a new target for treatment of hypertension. There have been no mechanistically novel treatments for this disease in the past 30 years. New classes of antihypertensive agents targeting CypD could add to the currently available therapeutic armamentarium to improve treatment of hypertension. Without being bound by mechanism or theory, the present inventors have discovered that specific CypD depletion in endothelial and smooth muscle reduces vascular oxidative stress, protects vascular relaxation and attenuates hypertension; CypD-K166 acetylation contributes to vascular dysfunction and hypertension; and CypD inhibition and blocking CypD hyperacetylation after onset hypertension improve vascular function.

The present invention meets a long-felt need because clinical data show that one third of adult population has hypertension and estimated 1.4 billion people have hypertension worldwide. This disease represents a major risk factor for stroke, myocardial infarction, and heart failure. Despite treatment with multiple drugs, third of hypertensive patients remain hypertensive, likely due to the mechanisms that are not affected by current treatments; however, there have been no mechanistically novel treatments for hypertension in the past 30 years. New classes of antihypertensive agents could therefore add to the currently available therapeutic armamentarium to improve treatment of hypertension.

Hypertension is a multifactorial disorder. However, in almost all experimental models of hypertension, production of reactive oxygen species (ROS: [symbol] and H₂O₂) is increased in multiple organs. In the brain, ROS promote neuronal firing, increasing sympathetic outflow. In the kidney, ROS act in multiple sites to promote sodium resorption and volume retention. In the vasculature ROS promote vasoconstriction and remodeling, increasing systemic vascular resistance. Our group has revealed several sources of ROS contributing to hypertension, including the NADPH oxidase, uncoupled nitric oxide synthase and the mitochondria and defined their interaction. ROS overproduction leads to oxidative stress which promote Target-Organ-Damage in hypertension. Antioxidant therapy is not currently available and common antioxidants like ascorbate and vitamin E are ineffective in preventing cardiovascular diseases and hypertension but therapies specifically targeted at mitochondria represent promising strategies to reduce target-organ-damage.

Mitochondrial dysfunction contributes to the pathogenesis of hypertension and cardiovascular disease; however, despite the central role of mitochondria in human health and disease, there are no approved drugs that directly target mitochondria. Mitochondrial dysfunction is characterized by impaired ATP production and increased oxidative stress leading to cell dysfunction and apoptosis. Mitochondrial permeability transition pore (mPTP) plays a key role in mitochondrial dysfunction and end-organ-damage in hypertension. The present inventors discovered that depletion or inhibition of Cyclophilin D (CypD), a regulatory subunit of mPTP opening, improves vascular function and attenuates hypertension. Previous studies implicated CypD in cell death and the present inventors showed that CypD is critical in vascular oxidative stress and endothelial dysfunction. The present inventors' data implicate a novel role of CypD acetylation and reactive isoLevuglandins (isoLG) in mPTP opening and vascular dysfunction.

The present inventors have previously reported that inhibition of CypD in mitochondria isolated from endothelial cells prevents superoxide ( [symbol] ) overproduction and in this work we propose that genetic deletion of vascular CypD or specific inhibition of CypD will decrease vascular oxidative stress, improve endothelial function and reduce hypertension.

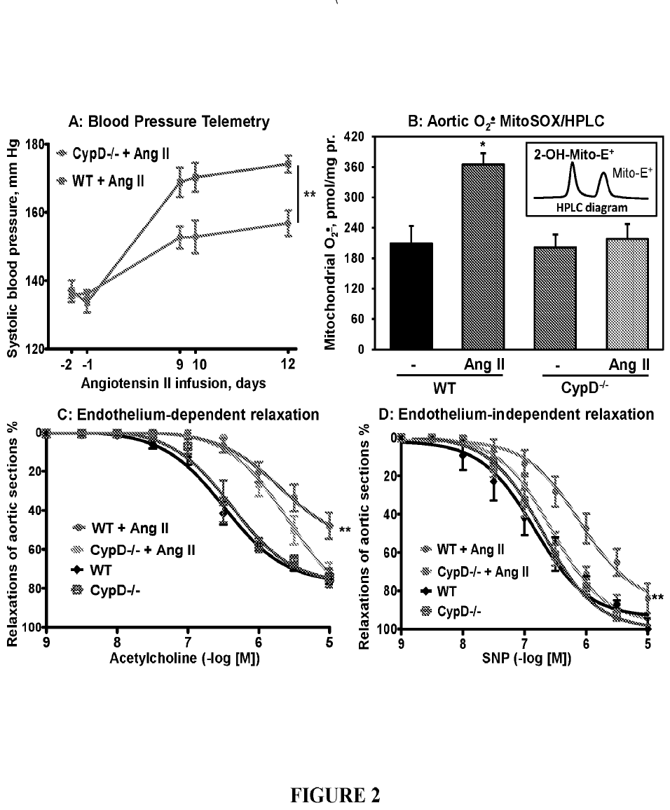

The present inventors have shown that CypD deficiency in CypD knockout mice (CypD^{-/-}) prevents overproduction of mitochondrial [symbol] in angiotensin II (AngII) infused mice (FIG. 2A), attenuates hypertension (FIG. 2B), improves endothelium-dependent and endothelium-independent vasodilatation (FIG. 2C, D) compared with wild-type C57Bl/6J mice.

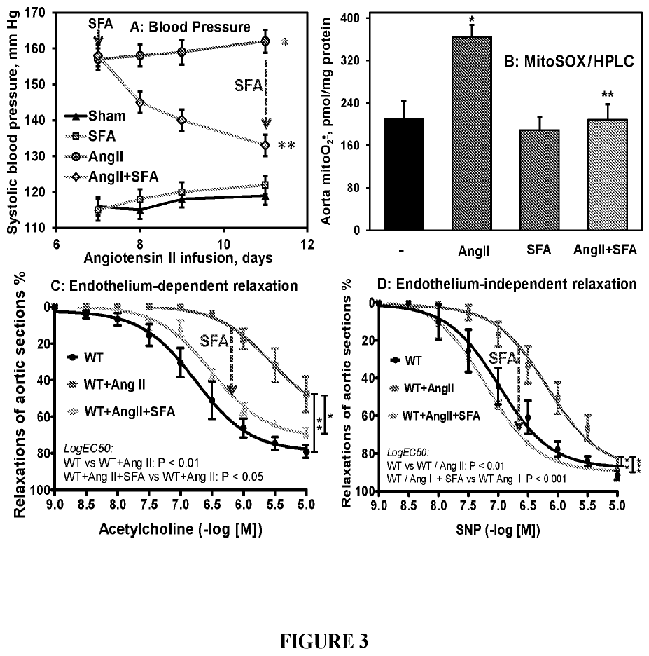

One aspect of the present invention is targeting CypD after onset of hypertension. The present inventors implanted wild-type mice with osmotic pump containing Ang II (0.7 mg/kg/day) and started treatment with Sanglifehrin A after onset of Ang II-induced hypertension (FIG. 3A). Indeed, treatment of hypertensive mice with CypD inhibitor Sanglifehrin A (i.p. 10 mg/kg/day) reduces blood pressure (FIG. 3A), normalizes mitochondrial [symbol] production (FIG. 3B) and improves vasodilatation (FIG. 3C,D).

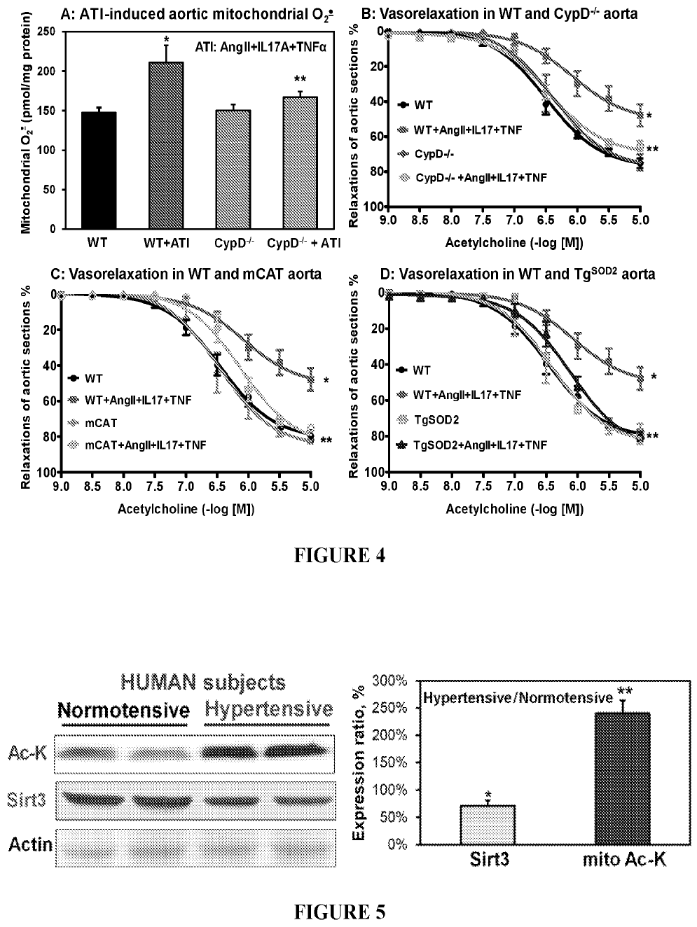

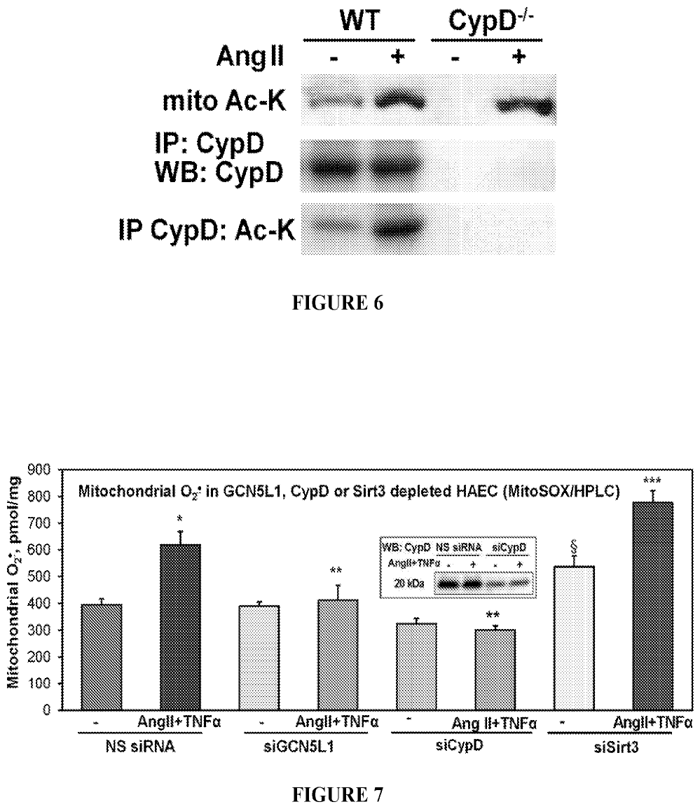

The present inventors have discovered that Angiotensin II and cytokines co-operatively induce CypD-dependent vascular dysfunction. IL17A and TNFα are required for AngII-induced hypertension. These cytokines are commonly associated with human hypertension and contribute to pathogenesis of this disease. The present invention shows that AngII, IL17A and TNFα co-operatively induce mitochondrial [symbol] in endothelial cells. The functional role of CypD-dependent vascular oxidative stress was tested in aortic sections isolated from mice overexpressing mitochondrial [symbol] scavenger SOD2 (Tg^{SOD2}) or mitochondria-targeted H₂O₂ scavenger catalase (mCAT). The inventors' data show that treatment of aortic vessels with AngII+IL17A+TNFα (ATI) leads to severe impairment of endothelial dependent vasodilatation which is prevented in aorta isolated from CypD^{-/-} mice. Interestingly, SOD2 overexpression or expression of mitochondria-targeted catalase significantly attenuated impairment of vasodilatation similar to the protection afforded by CypD deletion (FIG. 4).

These data show that pro-oxidant milieu of Ang II and cytokines leads to severe vascular oxidative stress, reduces endothelial NO and impairs vasodilatation, which is prevented by CypD depletion or scavenging of mitochondrial O₂\dot{—} and H₂O₂ in vessels from Tg^{SOD2} and mCAT mice. Furthermore, the inventors' data confirm an important role of CypD in regulation of vascular oxidative stress—the key role of CypD in stimulation of mitochondrial oxidative stress and vascular dysfunction in hypertension; however, the specific pathways of CypD activation are not clear.

The increase of hypertension with age is associated with decline of Sirt3 expression. The present inventors discovered that Sirt3 inactivation contributes to mitochondrial hyperacetylation in human hypertension. The present inventors analyzed Sirt3 expression and acetylation mitochondrial proteins in human subjects with essential hypertension. Western blots of peripheral blood mononuclear cells showed a 1.4-fold decrease in Sirt3 protein level and 2.6-fold increase in mitochondrial acetylation in hypertensive subjects (FIG. 5).

The present inventors recently reported mitochondrial hyperacetylation in human hypertension and mouse model of hypertension measured by mass spectroscopy and Western blot. Mitochondrial hyperacetylation in hypertension is accompanied by CypD acetylation which represents gain of function and promotes mPTP opening. To test this, the present inventors measured total acetylation of mitochondrial proteins and specific CypD acetylation in aortic mitochondria isolated from normotensive and hypertensive mice. Wild-type and CypD^{-/-} mice were infused with Ang II (0.7 mg/kg/day) or saline (vehicle) for 14 days, mice were sacrificed and mitochondria were isolated from aorta for Western blot studies. Western blot analysis showed robust increase in the total lysine acetylation (Ac-K ab) in mitochondrial lysate isolated from Ang II-infused hypertensive mice compared with sham wild-type mice. Following CypD immunoprecipitation Western blot did not show change in the CypD level (WT CypD) in normotensive and hypertensive mice; however, CypD acetylation was substantially increased in aortic mitochondria isolated from hypertensive mice. Note, CypD^{-/-} mice are protected from hypertension and endothelial dysfunction (FIGS. 2 and 4) and Western blot did not show CypD or CypD acetylation in samples from CypD^{-/-} mice confirming the specificity of Western blot.

The role of CypD acetylation in endothelial dysfunction is not clear. However, acetylation of lysine 166 is a gain of function which promotes mPTP opening and mitochondrial Sirt3 deacetylates CypD-K166. CypD inhibitors such as cyclosporine A bind close to K166 and prevent the CypD-mediated mPTP opening. In myocytes, mutation of K166 to arginine (CypD-K166R) mimics deacetylation and attenuates mPTP opening while mutation of K166 to glutamine (K166Q) mimics acetylation, enhances mPTP opening and exacerbates ischemia-reperfusion injury. GCN5L1 mediated acetylation counter-regulates the Sirt3 mediated deacetylation. The present inventors show that in endothelial cells GCN5L1 depletion reduces mitochondrial O₂^{•} while depletion of Sirt3 deacetylase enhances production of mitochondrial O₂^{•}. Human aortic endothelial cells (HAEC) were transfected with non-silencing siRNA (NS), GCN5L1 siRNA, Sirt3 siRNA or CypD siRNA. Three days after transfection cells were stimulated with Ang II plus TNFα, and O₂^{•} was measured by HPLC analysis of O₂^{•} specific product of MitoSOX, Mito-2OH-E⁺ It was found that GCN5L1 depletion abolishes the O₂^{•} overproduction similar to CypD depletion while Sirt3 depletion increased both basal and stimulated mitochondrial O₂\dot{—} (FIG. 7). These data support the role of CypD acetylation in endothelial oxidative stress.

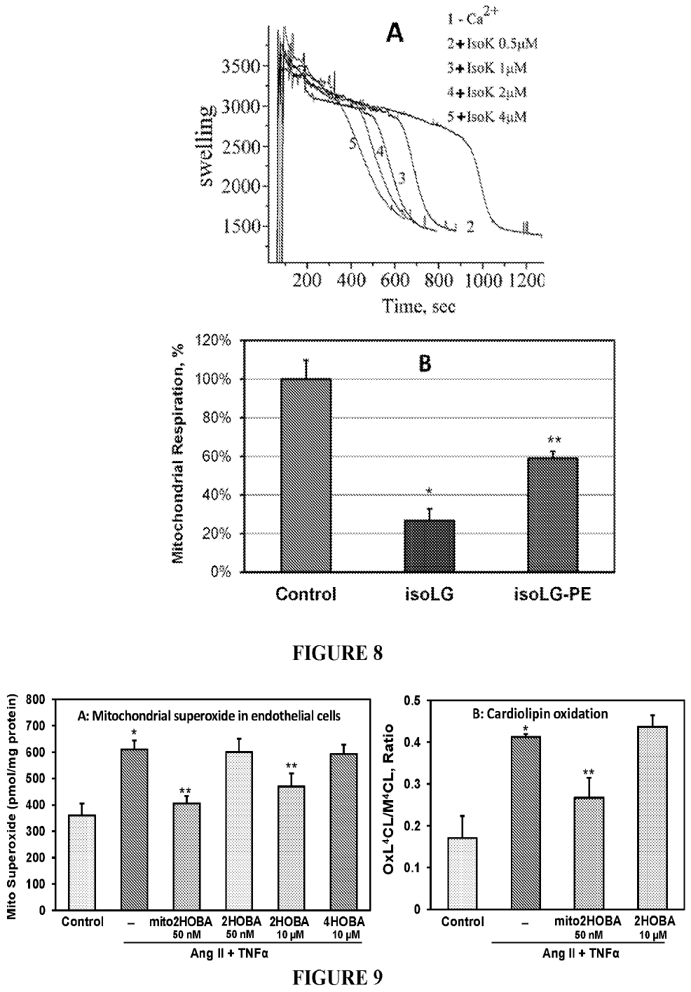

The present inventors have also learned that highly reactive lipid dicarbonyls derived from arachidonic acid, isolevuglandins (isoLG), are a mechanistic link between pathogenic reactive oxygen species and disease progression, and found acute isoLG exposure of mitochondria induces CypD-dependent mPTP opening and inhibits mitochondrial respiration (FIG. 8). Reactive isoLG produce protein-Lysine adducts and cytotoxic isoLG-phosphatidylethanolamine adduct (isoLG-PE) which can independently contribute to mitochondrial dysfunction. Indeed, acute treatment of isolated mitochondria with isoLG-PE inhibited respiration by 41% while similar dose of isoLG reduced respiration by 74% which support potential role of both isoLG-PE and isoLG protein-Lysine adducts in mitochondrial dysfunction.

The present inventors developed the mitochondria-targeted isoLG scavenger mito2HOBA by conjugating the lipophilic cation triphenylphosphonium to 2HOBA. The membrane potential of mitochondria within living cells is negative inside (−150 mV). As this membrane potential is much higher than in other organelles within cells, triphenylphosphonium lipophilic cations selectively accumulate in mitochondrial matrix by more than a five hundred-fold.

The present inventors discovered that Mito2HOBA reduces mitochondrial O₂^{•} production and inhibits cardiolipin oxidation, and show that mitochondrial oxidative stress produces isoLG and scavenging of isoLG improves mitochondrial function. Mitochondrial O₂^{•} and cardiolipin oxidation (specific marker of mitochondrial dysfunction and oxidative stress) in cultured human aortic endothelial cells (HAEC) were incubated with Ang II and TNFα. These agents were chosen in combination because we have shown they both contribute to endothelial dysfunction in hypertension. Ang II+TNFα induced mitochondrial oxidative stress as measured by 2-fold increase in mitochondrial O₂^{•} and 2.5-fold increase in cardiolipin oxidation. Treatment with mitochondria-targeted isoLG scavenger mito2HOBA (50 nM) diminished mitochondrial oxidative stress (FIG. 9A), while much higher concentrations of untargeted 2HOBA required to have similar protection. Mito2HOBA was more effective in prevention of cardiolipin oxidation than 2HOBA (FIG. 9B). These data support a previously unidentified role of isoLG in the mitochondrial dysfunction and demonstrate the feasibility of using very low doses of mito2HOBA for therapeutic purposes.

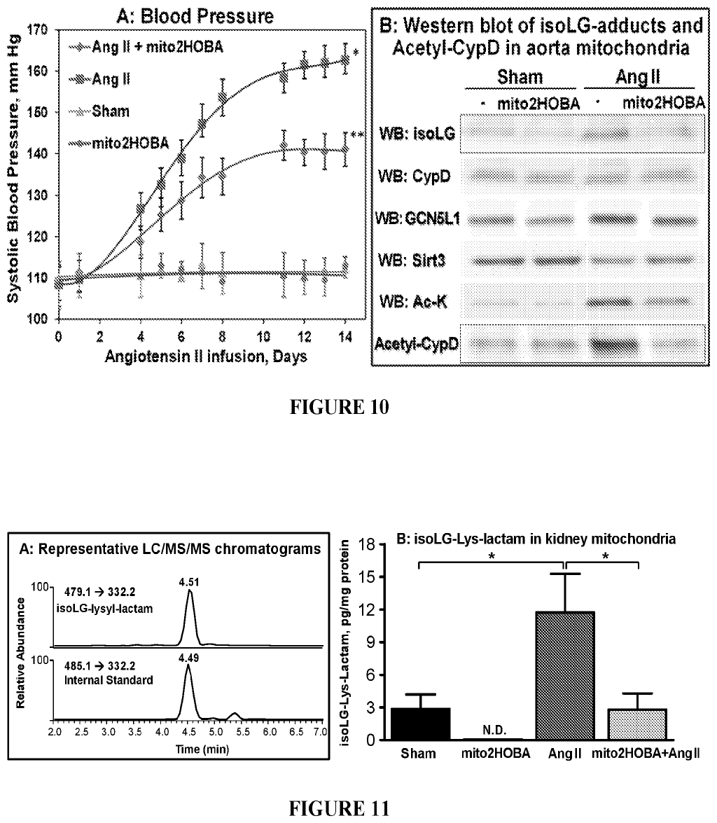

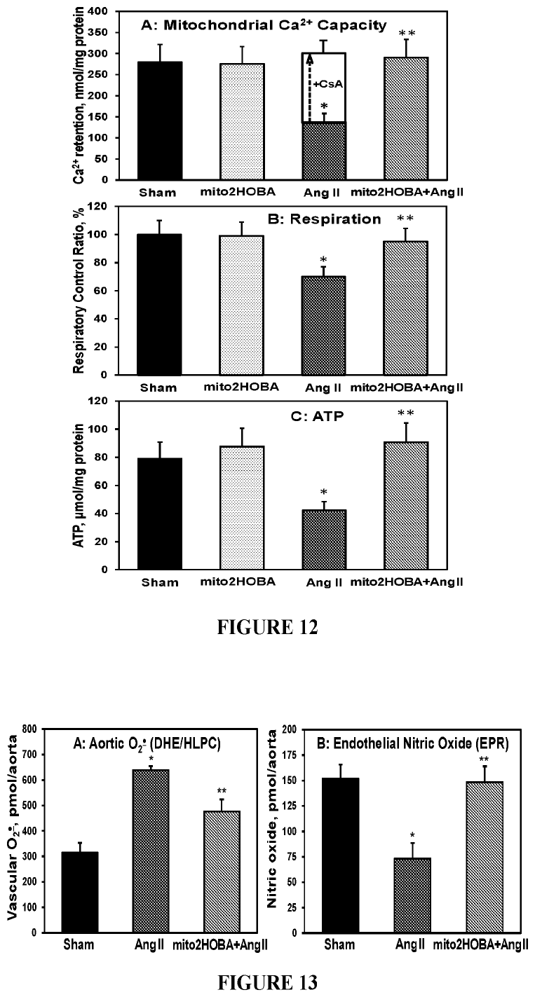

The present inventors show that Mito2HOBA attenuates hypertension, reduces mitochondrial isoLG and prevents CypD hyperacetylation, and that that treatment with mitochondria-targeted isoLG scavenger mito2HOBA reduces vascular oxidative stress, protects endothelial function and attenuates hypertension. Sham or Ang II-infused mice were supplemented with mito2HOBA in the drinking water (0.1 g/L) or plain water. Mito2HOBA significantly attenuated Ang II-induced hypertension (FIG. 10A). Following 14 days of Ang II infusion, mice were sacrificed, aortas were isolated for Western blot studies. Hypertension was associated with robust increase in mitochondrial isoLG adducts measured by D11 antibody, and mito2HOBA prevents accumulation of isoLG adducts. CypD expression did not changed; however, and acetyltransferase GCN5L1 was increased and deacetylase Sirt3 was decreased in hypertensive mice. This causes an imbalance between mitochondrial acetylation and deacetylation pathways leading to hyperacetylation of mitochondrial proteins measured by Ac-K and CypD hyperacetylation. Mito2HOBA corrects the imbalance between GCN5L1 and Sirt3, reduces mitochondrial Ac-K and prevents CypD hyperacetylation which implicates mitochondrial isoLG in CypD acetylation (FIG. 10B).

Mito2HOBA prevents Ang II-induced accumulation of mitochondrial isoLG-Lys-Lactam protein adducts, and the present inventors measured isoLG-Lysyl-Lactam adducts by liquid chromatography tandem mass spectrometry (LC/MS) after proteolytic digestion of extracted proteins as have been previously described. It was confirmed that hypertension was associated with 4-fold increase in the mitochondrial isoLG-Lysyl-Lactam protein adducts; and supplementation with the mitochondria-targeted isoLG scavenger mito2HOBA abolished isoLG-Lysyl-Lactam adducts formation in mitochondria (FIG. 11).

Hypertension impairs mitochondrial function and mito2HOBA attenuates mitochondrial dysfunction, and accumulation of mitochondrial isoLG in hypertension promotes CypD acetylation and mPTP opening, impairs mitochondrial respiration and reduces ATP. Scavenging of mitochondrial isoLG with mito2HOBA prevents these deleterious effects. The inventors analyzed kidney tissue isolated from control mice (Sham), mice drinking mito2HOBA, mice infused with Ang II and Ang II-infused mice supplemented with mito2HOBA (mito2HOBA+Ang II). Indeed, Ang II-infusion reduced Ca²⁺-retention capacity increasing mPTP opening, impaired mitochondrial respiration and reduced kidney ATP; however, mito2HOBA supplementation reduced CypD acetylation, attenuates mPTP opening, protects mitochondrial respiration and preserves normal ATP (FIG. 10, 12). These data show the pathophysiological role of mitochondrial isoLG in mitochondrial dysfunction and hypertension.

Thus, an embodiment of the present invention is Mito2HOBA compounds that reduce vascular oxidative stress and improve endothelial function. The present inventors were the first to show that vascular O₂^{•} overproduction contributes to endothelial dysfunction in hypertension. Among the other actions O₂^{•} inactivates endothelial nitric oxide (NO), promotes vasoconstriction and vascular remodeling and ultimately increases systemic vascular resistance. Decreased NO bioavailability is therefore a hallmark of endothelial oxidative stress in hypertension due to NO oxidation, reduced NO production and eNOS uncoupling. We measured aortic O₂^{•} using the fluorescent O₂^{•} probe DHE and HPLC as we described previously. Endothelial NO was quantified by Electron Spin Resonance (ESR) and specific NO spin trap Fe(DETC)₂. As shown in FIG. 13, the present inventors discovered that mito2HOBA reduces vascular O₂^{•} in Ang II-infused mice and preserves NO bioavailability. These data demonstrate the previously unrecognized role of mitochondrial isoLG in vascular oxidative stress and endothelial dysfunction.

### II. Vascular Oxidative Stress

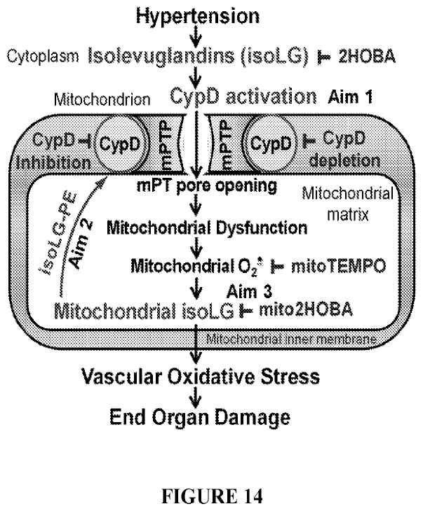

The present inventors have discovered that the mitochondria are dysfunctional in hypertension and have defined a novel role of mitochondrial oxidative stress in this disease. Mitochondria are the major source of superoxide radicals ( [symbol] ) and are rich in unsaturated fatty acids. Free radical oxidation of arachidonic acid produces highly reactive isolevuglandins (isoLG), which the present inventors have found to cause mitochondrial dysfunction by opening of the mitochondrial permeability transition pore (mPTP), and inhibition of the mPTP regulatory subunit cyclophilin D (CypD) reduces isoLG-induced mitochondrial dysfunction. Recently, we discovered that inhibition of mPTP opening by CypD depletion or CypD inhibition diminishes mitochondrial [symbol] , improves vascular relaxation and reduces hypertension. The present inventors also developed new mitochondria-targeted isoLG scavenger mito2HOBA compounds. This novel compounds reduce mitochondrial isoLG-protein adducts, inhibits oxidation of cardiolipin, a specific marker of mitochondrial oxidative stress, diminishes vascular [symbol] , normalizes endothelial nitric oxide and reduces hypertension. These data are in line with feed-forward stimulation of mitochondrial oxidative stress and show the therapeutic benefit of targeting mitochondrial isoLG in treatment of cardiovascular diseases. isoLG causes CypD-mediated mitochondrial dysfunction contributing to end organ damage, and that measures to reduce mitochondrial isoLG will diminish CypD activation and improve vascular function. This novel concept may lead to a paradigm-shift in defining mitochondrial isoLG as a new target in the treatment of cardiovascular diseases (FIG. 14).

Production of reactive oxygen species (ROS: O₂^{•} and H₂O₂) is increased in hypertension in multiple organs, including critical centers of the brain, the vasculature and the kidney. The present inventors have shown several sources of ROS contributing to hypertension, including the NADPH oxidase, uncoupled nitric oxide synthase and the mitochondria and defined their interaction. Meanwhile, antioxidant therapy is not currently available and common antioxidants like ascorbate and vitamin E are ineffective in preventing cardiovascular diseases and hypertension since these agents unlikely reach important sites of ROS production such as mitochondria. Additionally, the present inventors have discovered new isoLG-dependent mechanism responsible for mitochondrial dysfunction and end-organ-damage in hypertension. The compounds of the present invention target mitochondrial isoLG to diminish mitochondrial oxidative stress, improve vascular function and reduce hypertension.

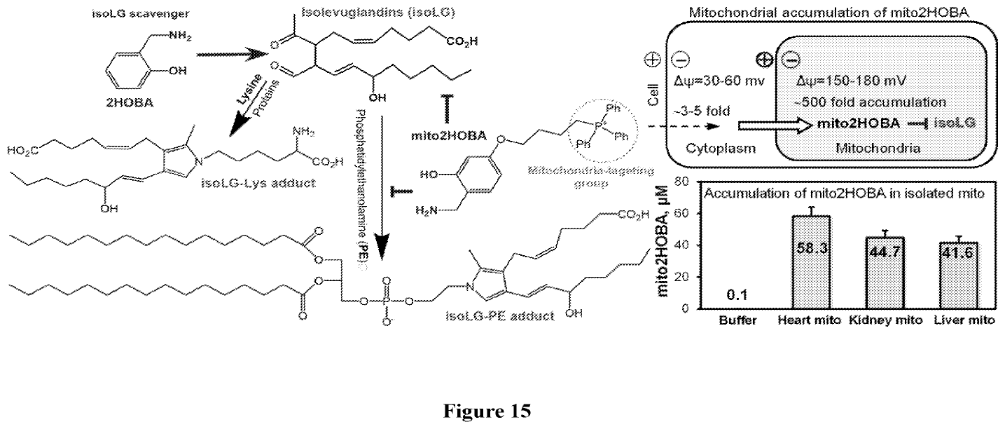

To demonstrate an example of scavenging isoLG in mitochondria to improve mitochondrial function mitochondria-targeted isoLG scavenger mito2HOBA compounds were developed. (See FIG. 15, for example). The membrane potential of mitochondria within living cells is negative inside (−150 mV). As this membrane potential is much higher than in other organelles within cells, lipophilic cations such as triphenylphosphonium (TPP) selectively accumulate within mitochondria. Molecules conjugated to TPP are therefore targeted to the mitochondria. As example, mitoTEMPO is concentrated within the mitochondrial matrix by more than a five hundred-fold.

The above example is a water soluble compound which can be supplied to cells in the media and provided to animals in the drinking water. In our preliminary in vitro and in vivo experiments mito2HOBA was well tolerated by cultured human aortic endothelial cells (HAECs) at concentrations up to 200 nM and when administered in the drinking water in doses from 0.1-0.3 g/Liter. Mass-Spec analysis of kidney and heart mitochondria isolated from mice received mito2HOBA with drinking water (0.1 g/Liter) for 5-days confirmed predominant accumulation of mito2HOBA in the mitochondrial fraction (by 80%) at μM levels. Likewise, incubation of isolated mitochondria with mito2HOBA (0.1 μM) causes robust accumulation of mito2HOBA in the mitochondrial pellet by 400 to 600-fold (FIG. 15, insert).

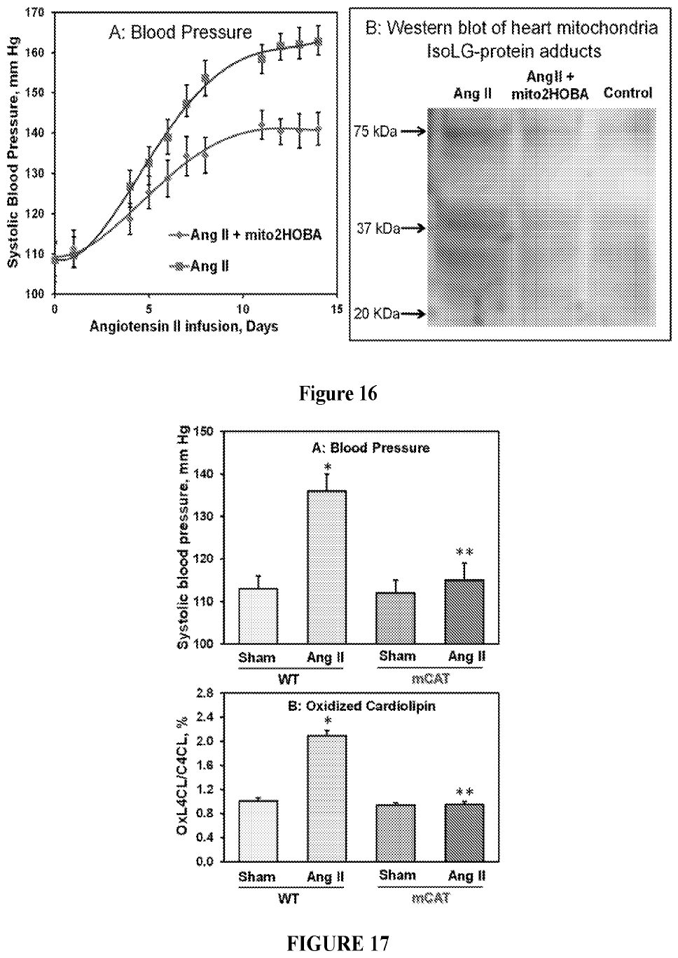

The present inventors implanted an osmotic minipump with Ang II (0.7 mg/kg/day) or saline (Sham) in C57Bl/6J mice receiving mito2HOBA in the drinking water (0.1 g/L) or plain water. It was found that mito2HOBA supplementation significantly attenuates Ang II-induced hypertension (FIG. 16A). Following 14 days of Ang II infusion, mice were sacrificed, hearts were isolated for mitochondrial studies and aortas were isolated for the analysis of vascular [symbol] and endothelial nitric oxide. As expected, hypertension was associated with increased formation of mitochondrial isoLG measured by Western blot analysis of heart mitochondria using D11 antibody. Furthermore, mito2HOBA reduced accumulation of isoLG adducts in the heart mitochondria of Ang II-infused mice (FIG. 16B).

To show that hypertension is associated with mitochondrial oxidative stress and that scavenging of mitochondrial H₂O₂ would reduce mitochondrial dysfunction and attenuates hypertension, the present inventors studied C57Bl/6J wild-type (WT) and transgenic mice expressing the mitochondria-targeted H₂O₂ scavenger, catalase (mCAT). Infusion with low dose of Ang II (0.3 mg/kg/day) increased blood pressure (136 mm Hg) in wild-type but not in mCAT (115 mm Hg) mice (FIG. 17B). Following 14 days of Ang II infusion, mice were sacrificed, hearts were isolated for measurements of the marker of mitochondrial oxidative stress, cardiolipin oxidation as we have previously described. As expected, hypertension was associated with increased cardiolipin oxidation in wild-type mice. Interestingly, cardiolipin oxidation was completely abrogated the in Ang II-infused mCAT mice (FIG. 17B).

These data support a previously unrecognized role of mitochondrial oxidative stress and mitochondrial isoLG in endothelial dysfunction and hypertension. As shown above, the present inventors show that CypD deficiency in CypD^{-/-} mice attenuates Ang II-induced hypertension, prevents overproduction of mitochondrial [symbol] , improves endothelium-dependent and endothelium independent vasodilatation compared with wild-type C57Bl/6J mice (FIG. 9).

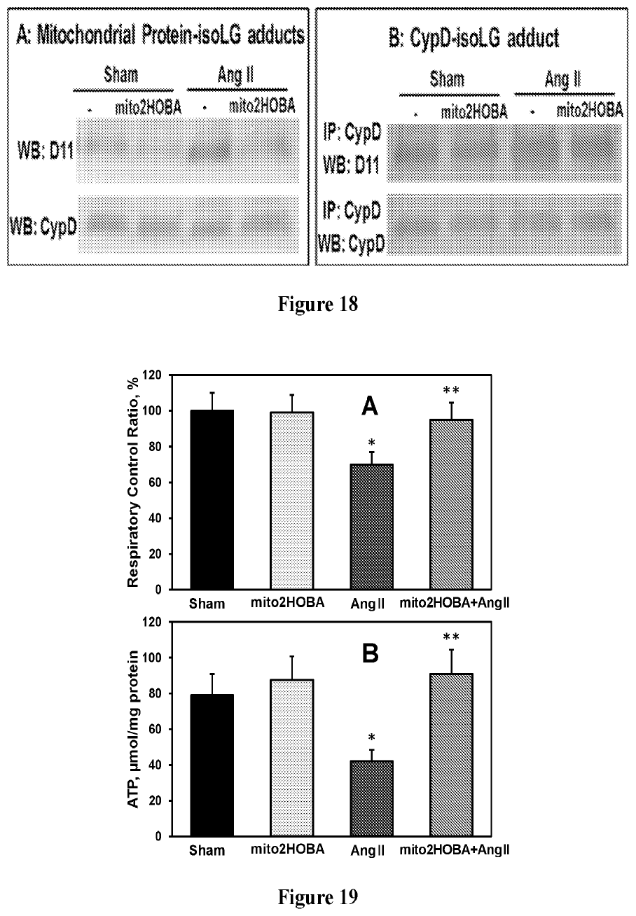

To define a potential role of CypD-isoLG interactions in mitochondria of vascular cells the present inventors isolated mitochondrial fractions from aortas dissected from mice infused with Ang II and measured isoLG-protein adduct formation using the anti-isoLG D11 antibody.³² Ang II-induced hypertension was associated with robust increase in mitochondrial isoLG in aorta and mito2HOBA supplementation attenuated accumulation of mitochondrial isoLG (FIG. 18A). The potential CypD-isoLG adduct formation was determined by CypD immunoprecipitation and Western blot with anti-isoLG D11 antibody. The data show that Ang II infusion increases both protein isoLG adducts and CypD-isoLG in the mitochondria and mito2HOBA diminished this (FIG. 18B).

Hypertension impairs mitochondrial function and mito2HOBA attenuates mitochondrial dysfunction. Based on above results the present inventors hypothesized that accumulation of mitochondrial isoLG in hypertension leads to impaired mitochondrial respiration and reduced ATP production, and scavenging of mitochondrial isoLG with mito2HOBA improves mitochondrial respiration and preserves ATP synthesis. To show this, the present inventors analyzed kidney tissue isolated from control mice (Sham), mice drinking mito2HOBA (mito2HOBA), mice infused with Ang II (Ang II) and Ang II-infused mice supplemented with mito2HOBA (mito2HOBA+Ang II). It was found that mitochondrial respiratory was impaired and kidney ATP level was significantly reduced in the kidneys of hypertensive Ang II-infused mice. Interestingly, mito2HOBA supplementation to Ang II-infused mice protects mitochondrial respiration and preserves normal ATP production (FIG. 19). These data show the pathophysiological role of mitochondrial isoLG in mitochondrial dysfunction associated with hypertension.

The invention thus being described, it would be obvious that the same can be varied in many ways. Such variations that would be obvious to one of ordinary skill in the art is to be considered as being bard of this disclosure.

Unless otherwise indicated, all numbers expressing quantities of ingredients, properties such as reaction conditions, and so forth used in the Specification are to be understood as being modified in all instances by the term “about.” Accordingly, unless indicated by the contrary, the numerical parameters set forth in the Specification and Claims are approximations that may vary depending upon the desired properties sought to be determined by the present invention.

Notwithstanding that the numerical ranges and parameters setting forth the broad scope of the invention are approximations, the numerical values set forth in the experimental sections or the example sections are reported as precisely as possible. Any numerical value, however, inherently contain certain errors necessarily resulting from the standard deviation found in their respective testing measurements.

1. A compound of the following formula: 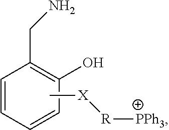
wherein:
X is a bond, —O—, or —CH₂—; and
R is C₁ to C₁₂ substituted or unsubstituted alkyl; and stereoisomers and pharmaceutical salts thereof.

2. A compound of the following formula: 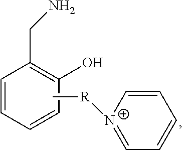
wherein
R is C₁ to C₁₂ substituted or unsubstituted alkyl; and stereoisomers and pharmaceutical salts thereof.

3. A compound of the following formula: 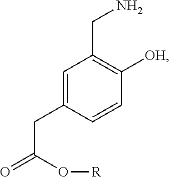
wherein
R is C₁ to C₁₂ substituted or unsubstituted alkyl; and stereoisomers and pharmaceutical salts thereof.

4. A compound of the following formula: 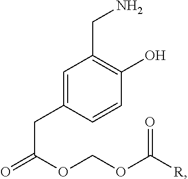
wherein
R is C₁ to C₁₂ substituted or unsubstituted alkyl; and stereoisomers and pharmaceutical salts thereof.

5. A compound of the following formula: 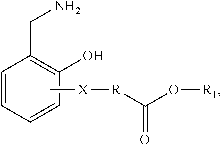
wherein
X is a bond, —O—, or —CH₂—;
R is C₁ to C₁₂ substituted or unsubstituted alkyl; and
R₁ is C₁ to C₁₂ substituted or unsubstituted alkyl or acetoxymethyl; and stereoisomers and pharmaceutical salts thereof.

6. A compound of the following formula: 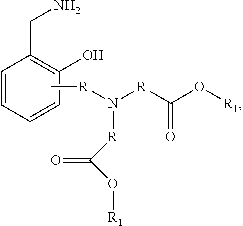
wherein
each R is independent and chosen from C₁ to C₁₂ substituted or unsubstituted alkyl; and
each R₁ is independent and chosen from C₁ to C₁₂ substituted or unsubstituted alkyl or acetoxymethyl; and stereoisomers and pharmaceutical salts thereof.

7. A compound of the following formula: 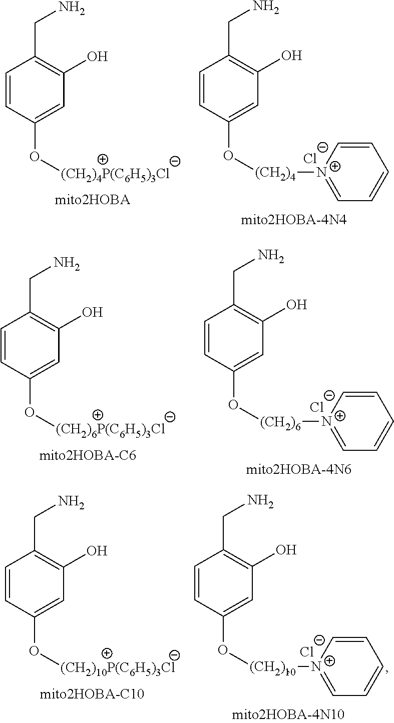
and stereoisomers and pharmaceutical salts thereof.

8. A compound of the following formula: 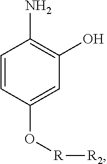
wherein
R is C₁ to C₁₂ substituted or unsubstituted alkyl; R₂ is selected from —P-Ph₃; or 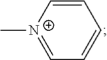
and stereoisomers and pharmaceutical salts thereof.

9. A method of treating, preventing, and ameliorating hypertension in a subject, comprising administering an effective amount of a compound of claim 1 or a pharmaceutically acceptable salt thereof.

10. A method of treating, preventing, and ameliorating vascular oxidative stress in a subject, comprising administering an effective amount of a compound of claim 1.

11. A method of treating, preventing, and ameliorating at least one of vascular oxidative stress, improve vascular functions and/or reduce hypertension, comprising administering to a subject a compound that targets mitochondrial CypD to inhibit vascular oxidative stress, improve vascular functions and/or reduce hypertension.

12-13. (canceled)

14. A method of treating, preventing, and ameliorating hypertension in a subject, comprising administering an effective amount of a compound of the following formula: 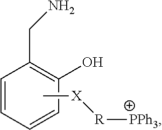
wherein:
X is a bond, —O—, or —CH₂—; and
R is C₁ to C₁₂ substituted or unsubstituted alkyl; and stereoisomers and pharmaceutical salts thereof.

15. (canceled)

16. A pharmaceutical composition, comprising a compound of claim 1; and a pharmaceutically acceptable carrier.
